(19) United States
(12) Patent Application Publication
(10) Pub. No.: US 2025/0302644 A1
(43) Pub. Date: Oct. 2, 2025

(54) Ankle-Foot System with an Energy Storing Keel, Vertical Shock Absorbing Pylon, Active Dorsiflexion and Axial Rotation

(71) Applicant: Impulse Technology LLC, State College, PA (US)

(72) Inventors: Md Amanul Haque, State College, PA (US); Cody J. Vassiliou, State College, PA (US); Mst Kamrunnahar, State College, PA (US)

(21) Appl. No.: 19/082,440

(22) Filed: Mar. 18, 2025

Related U.S. Application Data
(60) Provisional application No. 63/569,879, filed on Mar. 26, 2024.

(51) Int. Cl. A61F 2/66; A61F 2/50

(52) U.S. Cl. CPC A61F 2/6607; A61F 2002/5003; A61F 2002/6692

(57) ABSTRACT

Embodiments can relate to a prosthetic foot system. The system can include an ankle joint housing co-locating a rotation sub-assembly, a torsional shock absorbing sub-assembly, and a vertical shock absorbing sub-assembly. The system can include a foot component attached to the ankle joint housing. The system can be configured as a co-designed architecture to functionally integrate at least two functions of: (i) torsional shock absorption, (ii) multi-axial motion with stiffness modulation in single gait cycle, (iii) active dorsiflexion, and (iv) vertical shock absorption by causing the at least two functions to operate in concert.

### CROSS-REFERENCE TO RELATED APPLICATIONS

This patent application is related to and claims the benefit of priority of U.S. provisional patent application No. 63/569,879, filed on Mar. 26, 2024, the entire contents of which is incorporated herein by reference.

### FIELD OF THE INVENTION

The invention relates to adapters or components in lower limb prostheses that allow certain range of motion to effectively act as a prosthetic ankle and/or foot. The invention is a lower limb prosthesis with co-designed architecture, where multiple functions are synergistically integrated. It is an add-on component to be located under the pylon, thereby joining an ankle and a foot to a leg or pylon. It thus also falls in the category of prosthetic ankle-foot, where the ankle adapter is integrated with the foot.

### BACKGROUND OF THE INVENTION

Lower limb amputees require a prosthetic foot to regain basic ambulation ability. Below the knee lower limb prosthetics typically have 3 major components, the foot, the ankle, and the pylon, with the ankle being optional. Additional components/features such as shock absorption and active dorsiflexion can increase comfort and decrease probability of tripping over respectively.

Prior art is to embody these functions in physically different modular components and then assemble them together. Each module would have one function. For example, shock absorber unit (linear motion) is housed in the pylon component whereas torsional (rotational motion) shock absorber is added on top of it as a separate unit. In a similar manner, active dorsiflexion function is added via the ankle component. To mimic the functioning of a human ankle-foot, current art requires the prosthetists to add one module with or over another. This is merely physical assembly and not the intelligent integration of these functional components. As a result, the total ankle-foot product becomes heavy and tall in size. Product weight is one of the most critical parameters in lower limb prosthesis. Product height is also very important because too high build height may not fit most of the amputees. Therefore, a new ankle-foot design philosophy is essential to provide all the above-mentioned functionality in a very small and light product.

FIG. 1a shows an example of our new design philosophy towards ankle-foot design, where the functionalities are both integrated and co-located in the same component housing; this leads to the smallest size and weight. Instead of just stacking components together, the functions are intelligently implemented by concentric layers or compartments. This is indicated by the dashed vertical lines, which mean that the components are not physically separate, rather can be co-located in the same space. FIG. 1b shows an example of existing design philosophy in prior art, where the functionalities are typically stacked on top of each other in separate housings connected by additional mounting mechanisms; this adds unnecessary size and weight.

FIG. 2, containing preferred embodiment 1, shows a high-level overview of how these functionalities can be integrated and co-located within the integrated ankle joint housing 2. A vertical shock absorbing sub-assembly 3 can be co-located. Vertical shock absorption refers to the axial motion in the pylon with respect to the foot, which absorbs shock in the limb during landing through the stance phase of gait. It helps lower socket pressure, thereby reducing discomfort and pain. An active dorsiflexion and multi-axial rotation sub-assembly 4 can be co-located. This refers to the automatic dorsiflexion motion of the ankle in the air during the swing phase of the gait to aid the user. It is critical to avoid accidental scraping of the toe component. If the total range of motion also covers one or two more planes beyond the plane of walking, it is labeled as multi-axial ankle motion, such as in the embodiment shown. A torsional shock absorber sub-assembly 5 can be co-located. Torsional shock absorption refers to the rotational motion of the ankle relative to the foot that helps absorbing torsional shock. This aids in walking on uneven terrain or walking with sharp twists and turns. Another core component of the ankle-foot product is the foot itself. The integrated prosthetic foot component 6 can enable advanced ambulation ability through an energy storing J-shaped keel system which allows for adaptation to uneven terrain and stronger toe push-off.

FIG. 3, containing preferred embodiment 7, shows another high-level overview of how these functionalities can be arranged within an integrated ankle joint housing 8. A vertical shock absorbing sub-assembly 9, active dorsiflexion and multi-axial rotation sub-assembly 10, torsional shock absorber sub-assembly 11, and integrated prosthetic foot component 12 can all be co-located.

We now present prior art to claim that existing intellectual properties are either single function or multi-functions, but one component stacked on the other rather than integrated co-location. U.S. Pat. No. 6,482,236B2 discloses a prosthetic ankle joint mechanism. It allows motion in the sagittal plane and features active dorsiflexion; different dorsiflexion and plantarflexion responses are achievable by adjusting the position of the retainer element. It does not contain a torsional shock absorbing unit or a vertical shock absorber unit.

U.S. Pat. No. 9,289,316B2 discloses a quasi-active prosthetic joint system. This innovation describes a series of springs, clutches, actuators, and sensors to modulate stiffness during sagittal rotation. Different springs will activate depending on the physical state of the joint. This system utilizes an active microcontroller with sensor feedback to respond in a multitude of ways depending on the physical state of the device. It does not contain a torsional shock absorbing unit or a vertical shock absorber unit.

U.S. Pat. No. 11,529,246B2 discloses a prosthetic ankle and foot combination. It achieves sagittal rotation and active dorsiflexion via a microcontroller. It does not contain a torsional shock absorber or vertical shock absorber unit. The ankle is described with variable, hydraulically dampening resistances, with independent resistances of plantar-flexion vs dorsiflexion. It includes an electronic control system to adapt the resistances dynamically.

U.S. Pat. No. 10,857,008B2 discloses an adapter for self-alignment in 3 dimensional planes for passive prosthetics. This describes a multi-axial ankle joint device, to be attached to an existing prosthetic foot. It has an axial shock absorbing unit, multi-axial motion in all 3 planes of motion, and a torsional shock absorbing unit, but does not include active dorsiflexion. The design is meant to be an add-on to an existing foot system rather than full functional integration. It is designed to grant real-time non-linear stiffness in all three planes of motion. Such multi-axial motion allows it to self-align on uneven terrain.

U.S. Pat. No. 10,390,974B2 discloses a prosthetic foot with removable flexible members. This invention describes a prosthetic foot/ankle assembly that provides variable stiffness. It has sagittal rotational motion, a vertical shock absorbing unit, and active dorsiflexion, but does not have a torsional shock absorbing unit. This stiffness profile can be manually set/activated, or also via some sort of powered actuator. One or more connecting members can be used depending on the level of stiffness desired, essentially making it somewhat modular to let a clinician fit the needs of a patient.

U.S. Pat. No. 8,317,877B2 discloses a prosthetic foot. This features a shock absorbing foot, but it does not have a torsional rotation unit or active dorsiflexion. It also provides energy transfer due to its shape and preferred construction material of carbon fiber, behaving like a spring. Both the heel and toe will absorb some energy during bending and release as gait progresses to help propel the user forward. The plates are connected via small bumpers, and the largest amount of energy can be stored by maximizing the length between the bumpers.

U.S. Pat. No. 7,942,935B2 discloses a device and system for prosthetic knees and ankles. This invention has active dorsiflexion but no vertical shock and torsional shock absorption. It describes a prosthetic ankle or knee with an articulating joint, though primarily an ankle joint is described and embodied within the images. The articulating joint consists of a rotary hub along with one or more vanes to facilitate fluid flow, essentially forming the basis for a hydraulic ankle. When the foot is in mid-air (“stance phase”), the hydraulic joint will automatically rotate to raise the toe of the attached foot, which is helpful for preventing tripping while ambulating. This, like many other ankle joint inventions, attempts to mimic the behavior of the biological ankle.

Published US Patent 20230126674A1 discloses a prosthetic foot/ankle system with automatic alignment. This is an ankle-foot prosthesis with active dorsiflexion and sagittal rotation. It does not have a vertical shock absorber or torsional shock absorber. A hydraulic damper and energy storing springs allow the ankle joint to rotate similarly to a biological ankle. It will adapt and align to inclines and the state of gait by modulating its stiffness. The system can automatically lock the joint in place in dorsiflexion and unlock when dorsiflexion loading has ended. A series of check valves and pistons enables this behavior.

U.S. Pat. No. 8,721,737B2 discloses a passive ankle prosthesis with energy return simulating that of a natural ankle. This device has active dorsiflexion, sagittal rotation, and a vertical shock absorber. It does not have a torsional shock absorber, and the implementation is bulky rather than a lean, functional integrated design. The invention describes an at least two degree of freedom, passive, ankle joint comprising primarily of compression springs. It claims that one of the degrees of freedom allows a lower leg component to compress slightly during ambulation, while a distinctly different second degree of freedom allows rotation about the joint. The energy stored along the first degree of freedom is released into the second degree of freedom. Therefore, it claims to release more energy than initially stored in the ankle deflection, claiming active behavior, which is typically only achieved through external power sources such as motors or actuators, from purely passive elements. The springs and axles are arranged with a series of slots to control the movement in the intended direction.

U.S. Pat. No. 7,819,926B1 discloses a prosthetic foot and ankle. This is an ankle-foot patent with sagittal rotation. It does not have a torsional shock absorber or a vertical shock absorber. The ankle and foot are coupled by an arch suspension yoke. The ankle allows for some articulations of the entire assembly. Additional leaf springs and elastomer springs enable additional rotation throughout the foot and ankle. It is constructed in a way that does not resemble a typical carbon fiber prosthetic foot; this device has what would typically be considered the “aesthetic foot shell” integrated as part of its design.

U.S. Pat. No. 8,480,760B2 discloses a passive ankle-foot prosthesis and orthosis capable of automatic adaptation to sloped walking surfaces and method of use. This is a passive ankle foot device with sagittal rotation and active dorsiflexion. It does not have a vertical or torsional shock absorbing unit. The system lets an amputee cross inclined or declined surfaces without losing balance via a series of cams and joints to emulate gait. The system self-adapts to change the torque moment depending on the state of the device and the surface topography it is on. It is light and easy to manufacture. It may be used as either a prosthesis or an orthosis.

U.S. Pat. No. 11,540,929B2 discloses a tapered flex plate for a prosthetic foot. This is a prosthetic foot design. It does not have an ankle joint providing active dorsiflexion, sagittal rotation, torsional shock absorbers, or vertical shock absorbers to improve rollover and performance, a lower foot member extends from the heel all the way to the toe. A second and optionally third member are stacked on top of the first at shorter overall lengths. The thickness of this second member is tapered such that it decreases in thickness as it gets closer to the toe. The thickness of the optional third member is tapered such that it decreases in thickness as it gets closer to the distal end of the heel. A gap may be present between the second and third members that closes during dorsiflexion, providing dynamic stiffness control.

U.S. Pat. No. 7,572,299B2 discloses a prosthetic foot with energy transfer. This invention is for a prosthetic foot or in some embodiments, an ankle-foot. The ankle-foot embodiment has sagittal rotation and active dorsiflexion, but not a torsional or vertical shock absorbing unit. Utilizing hydraulic pistons, it provides a variable stiffness response between the first and additional members via a small piston. The piston chamber contains a variable viscosity fluid to vary the ability of it to flow through at least one aperture.

U.S. Pat. No. 9,439,786B2 discloses a prosthetic ankle module. This is for a prosthetic ankle joint that can be arranged in multiple ways to provide one or more of the following: sagittal rotation, active dorsiflexion, and vertical shock absorption. It does not feature torsional shock absorbers. It includes a four-bar linkage assembly, and this assembly can be arranged in different configurations, such as in parallel or non-parallel, to provide functions the relevant functions.

U.S. Pat. No. 9,668,887B2 discloses a foot prosthesis with resilient multi-axial ankle. This is a prosthetic ankle-foot. The ankle can provide sagittal rotation but does not provide torsional shock absorption or vertical shock absorption. In one embodiment, it contains a soft heel, stabilization at heel strike, progressive stiffness at heel strike and toe off, smooth rollover, guided rollover, progressively increasing support form mid stance through toe off, natural feeling toe off, variable stiffness during rollover and a reduction in stresses in members that secure various foot components to one another. This is accomplished by a series of plates and stiffeners placed at strategic locations.

U.S. Pat. No. 8,888,864B2 discloses an energy storing foot plate. This invention is a prosthetic ankle joint that can be mounted to a foot plate to allow sagittal rotation and active dorsiflexion, but not a vertical shock absorber or torsional shock absorber. The ankle joint has a clevis joint that allows flexional rotation. The coupling structure will raise the toe during swing phase, commonly referred to as active dorsiflexion.

U.S. Pat. No. 450,297 discloses an artificial limb. This invention is of an early ankle-foot prosthesis design that has sagittal rotation and active dorsiflexion. The included pylon implementation allows for some shock absorption, but not torsional shock absorption, and is bulky. It features a toe joint in the foot that is allowed to rotate the toe up or down, as well as an ankle joint that is allowed to rotate up or down. A strap will attempt to pull the foot upwards during the act of stepping, an early form of what is today known as active dorsiflexion. The device is primarily made of wood, and describes some methods used to facilitate an easy repair of the device via detachable joints.

U.S. Pat. No. 1,357,074 discloses an artificial limb. This invention is of an early artificial ankle-foot that features sagittal rotation and vertical shock absorption, but not torsional shock absorption or active dorsiflexion. It features a shock absorbing component that will allow the ankle joint to move relatively vertically on load application. The arrangement of joints additionally allows for some plantarflexion and dorsiflexion on load application.

U.S. Pat. No. 2,594,945 discloses an ankle joint for artificial legs. This invention is of an early artificial ankle that provides vertical shock absorption and sagittal rotation, but not torsional shock absorption or active dorsiflexion. It describes a long-term prosthetic ankle that will provide cushioning, flexibility, and natural foot movements. The cushioned joints allow for a lighter weight without sacrificing flexibility. The joint is attached via screws, and the cushioned material along with strategically placed gaps in the assembly allow for the flexible motion of the ankle.

### SUMMARY OF THE INVENTION

GoraliPaa SFT (Shock Flex Torsion) is an ankle-foot product that embodies a new design philosophy of ‘co-designed architecture’. Here, multiple lower limb prosthesis functions are designed to co-exist in a synergistic or integrated manner. These prosthesis function can include vertical shock absorption, torsional shock absorption (axial rotation), multi-axial motion with stiffness modulation in single gait cycle, active dorsiflexion during swing phase and an energy storing and releasing foot. In comparison, the current art is ‘modular architecture’ where prosthesis components of various functions are physically assembled together. This invention reduces weight and build-height without sacrificing functionality or durability. The invention is not a logical deduction from the prior art since it demonstrates the claimed co-location of multiple functions leading to a superior outcome for amputees.

We provide an ankle and foot prosthesis for a prosthetic leg assembly that enables advanced ambulation capabilities while maintaining a short build height and light weight. The innovation is the ‘functional integration’ or ‘co-location’ of various functions (active dorsiflexion, vertical shock absorption, torsional shock absorption, and multi-axial rotation joint on an energy storing foot). For example, FIG. 2 showed how the physical mechanisms that give different functions are co-located in the same space, but in layers, like an onion. As described in detail below, the design philosophy is not present in prior art and the embodiments claimed also cannot be derived from prior art.

The ankle joint enables additional sagittal rotation (dorsiflexion and plantarflexion) as well as axial rotation (medial and lateral rotation) of the foot relative to the ankle. In particular, the axial rotational stiffness is differential, such that the stiffness will increase as the angle of rotation increases. Additionally, the sagittal rotation mechanism contains a compliant spring that is easy to overcome by the patient's body load; however, during swing phase, when the foot is in air, the spring will activate and rotate the foot upwards.

The assembly is a passive, solid-state mechanism, and therefore of lighter weight and smaller size than active mechanisms which require power sources and other additional components. This also means any prosthetic assembly featuring this mechanism will require little prosthetist or user attention regarding fitting and cleaning.

The prosthetic foot is of a particular shape such that it deflects and stores energy during early stages of gait to release it at a later stage of gait. It works in tandem with the integrated ankle joint to provide dynamic stiffness depending on the physical state of the foot, where the ankle joint can activate at a certain level of deflection of the foot to significantly increase the stiffness, which would be helpful during intensive activities.

Following are the attributes of the innovation:
a. The product is a biomimetic ankle joint and prosthetic foot combination product (commonly referred to as “ankle-foot”), to be attached to a lower limb prosthetic assembly to form a prosthetic limb. It is intended to be mounted under a pylon assembly, which is mounted to a socket holding the patient's residual limb.
b. The product is completely passive, containing no sensors, actuators, or electronics. There are no liquids or other non-solid-state components. This minimizes any maintenance required. Additionally, the components can be manufactured of materials that will not rust or otherwise significantly degrade in contact with water. These components could consist of a carbon fiber prosthetic foot, titanium ankle joint components, thermoplastic polyurethane bumpers, and stainless steel or otherwise treated fasteners.
c. The ankle joint is integrated with the prosthetic foot, and contains a vertical shock absorber, sagittal rotational capabilities (dorsiflexion and plantarflexion), automatically raises the toe during swing phase (foot in air), and differential axial rotation. These components are concentric and stacked like layers of an onion. The center of a pylon on the top of the ankle joint mates with a hole in the center of all the subsequent interior components. The interior components of the ankle joint will all slide up and down to accommodate the shock absorbing capabilities.
d. The ankle joint is allowed to rotate via a bumpered pin and hole joint on each lateral side. This enables sagittal rotation (dorsiflexion and plantarflexion). A compliant spring will automatically rotate the ankle joint to raise the prosthetic foot's toe while the invention is in swing phase (in air), which helps prevent patients from scraping the toe on the ground and stumbling like a biological ankle would. This spring is weak enough to be overcome by the patient's body weight during heel strike, and will provide some level of resistance and damping, tunable to body weight.
e. The ankle joint is allowed to rotate axially via a set of uniquely shaped torsional springs. In one embodiment, 3 springs can be used, with the first being the tallest, progressively decreasing in height until the final spring. Additionally, the cutout that mates with the pylon can be different for each spring, such that certain springs will only activate depending on the physical state of the assembly. (Example, from 0-5 degrees axial rotation, only one spring is activated, and the stiffness is at its lowest. From 5-10 degrees axial rotation, two springs are activated, and the stiffness is at a median level. From 10-15 degrees axial rotation, all 3 springs can be activated, and the stiffness will be at its highest level.) Since the third spring is shorter than the second, which is in turn shorter than the first, the additional stiffness provided by each spring will be less and less, such that the stiffness will not reach an uncomfortable level. This contributes to a differential, dynamically altered stiffness.
f. The curvature of the prosthetic foot has a favorable interaction with the ankle joint. The ankle joint contains a piece that under no load is not in contact with the prosthetic foot. This piece will make contact with the prosthetic foot, significantly shortening the effective cantilever arm, which significantly increases the stiffness of the prosthetic foot itself. This contributes to a dynamic sagittal load vs angle response curve. This piece can be tuned to activate under certain amounts of load, such as activating towards the end of every step, or only during heavier activities such as running and jumping which would cause greater than average deflection of the prosthetic foot compared to simply walking.
g. The prosthetic foot portion of the innovation is energy storing. Each of the two carbon fiber (or similar material) will bend and store potential energy which will then be released as kinetic energy later in the gait cycle. During the heel strike, the heel will bend and store energy to then unbend and be released into mid-stance and foot flat. Similarly, during mid-stance, the J-shaped keel will bend significantly and store energy to then unbend during toe-off and propel the patient forward.
h. An elastomeric material can be placed between the two prosthetic foot plates, which will provide additional dampening and resistance.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIGS. 1a and 1b show the design philosophy of the innovation and the prior art, respectively.

FIG. 2 is a high-level side cross-sectional view of one preferred embodiment revealing how the components can be intelligently co-located and integrated.

FIG. 3 is a high-level side cross-sectional view of another preferred embodiment revealing how the components can be intelligently co-located and integrated.

FIG. 4 is an isometric perspective view of the preferred embodiment from FIG. 2.

FIG. 5 is a top perspective view of the preferred embodiment from FIG. 2.

FIG. 6 is an exploded isometric perspective view of the preferred embodiment form FIG. 2.

FIG. 7 is a front cross-sectional view of the preferred embodiment from FIG. 2.

FIG. 8 compares the shock absorber in an uncompressed due to no load state, 8a, and compressed due to vertical body load state, 8b.

FIG. 9 compares the sagittal rotation of the device when either no load is applied or a dorsiflexion load is applied 9a, and when a plantarflexion load is applied 9b.

FIG. 10 compares one of the torsional springs when a medial axial load is applied 10a, no axial load is applied 10b, and when a lateral axial load is applied 10c.

FIG. 11 compares the physical state of the device when a medial axial load is applied 11a, no axial load is applied 11b, and when a lateral axial load is applied 11c.

FIG. 12 compares the cutouts on each of the three torsional springs in FIGS. 12a, 12b, and 12c.

FIG. 13 shows the state of the device during swing phase, where the vertical shock absorber is uncompressed, active dorsiflexion causes the foot to be dorsiflexed, and the axial rotation angle is at zero degrees.

FIG. 14 shows the state of the device during an example heel strike, where the vertical shock absorber is slightly compressed, the foot is plantarflexed, and the axial rotation angle is medially rotated.

FIG. 15 shows the state of the device during an example mid-stance to toe-off, where the vertical shock absorber is significantly compressed, the foot is dorsiflexed, and the axial rotation angle is laterally rotated.

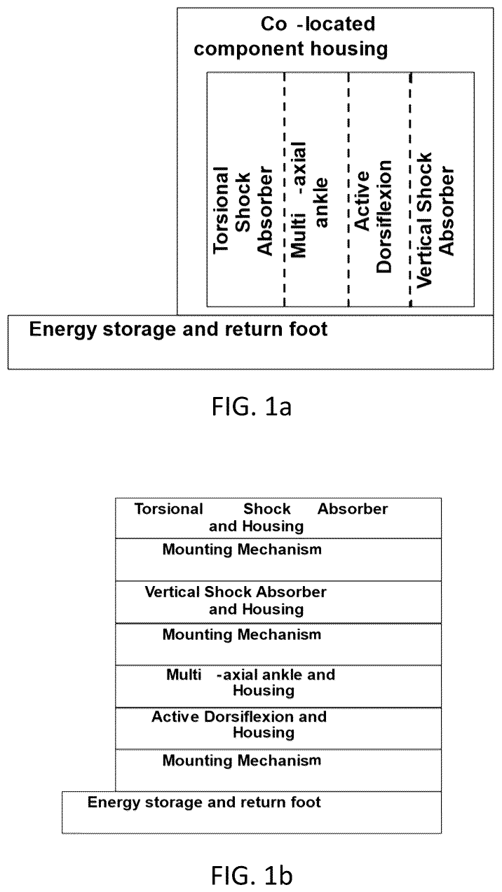

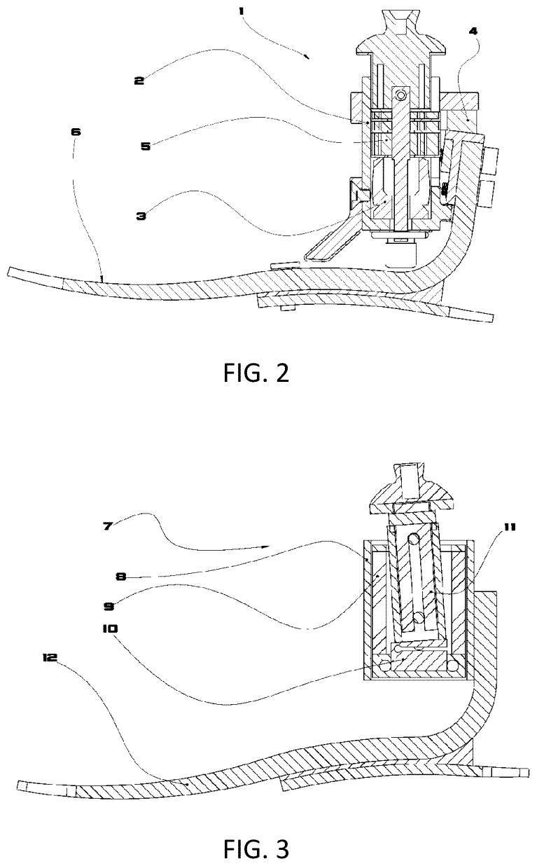

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

This innovation relates to GoraliPaa SFT, a foot-ankle system comprising of an energy-storing keel, an integrated shock absorbing pylon, an articulating ankle component that enables multi-axial motion with stiffness modulation in single gait cycle and active dorsi-flexion during swing phase, and a torsional component that enables ab/ad-duction. It is intended to be used with a cosmetic foot shell.

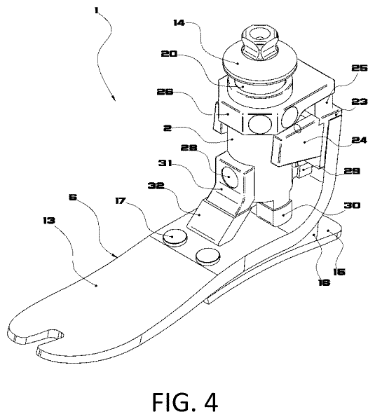

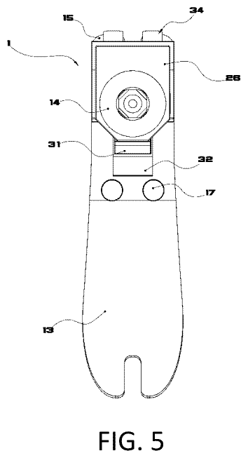

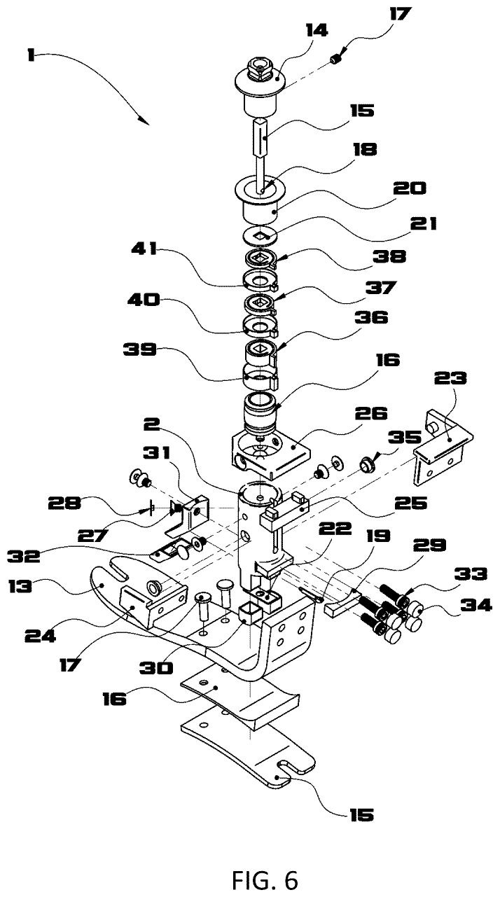

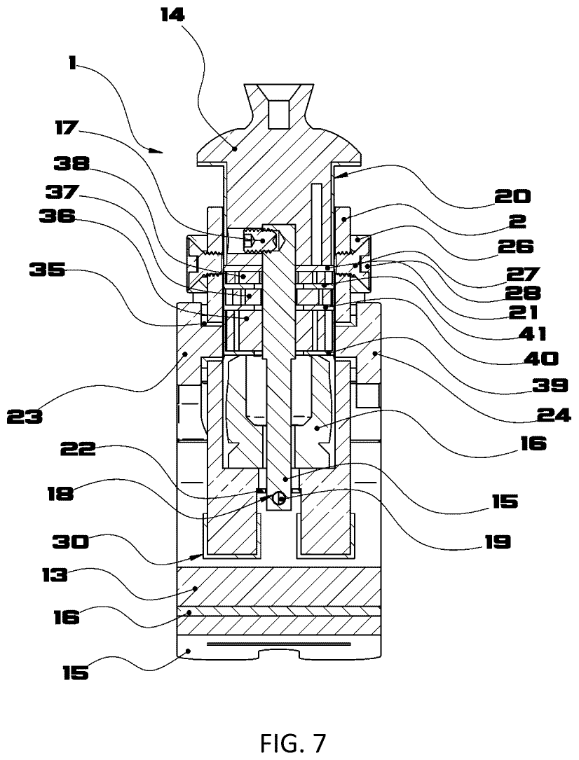

The embodiments contain 3 distinct functionalities, and all the components are integrated as a single product via a singular ankle joint housing 2. In FIGS. 4, 5, 6, and 7, relating to embodiment 1, the prosthetic foot system 6 contains an energy storing J-shaped keel 13 that is integrated with a vertical loading pylon 14 to as part of shock absorption sub-assembly 3. A heel component 15 is attached to the J-shaped keel section 13 along with an elastomer bumper 16 via one or more fasteners such as rivet 17 to form the prosthetic foot system 6. The vertical loading pylon 14 allows a controlled motion for shock absorption of up to 10 millimeters of vertical displacement via an extension rod 15 and a shock absorbing compression spring 16. The extension rod 15 may be attached to the vertical loading pylon 14 via a fastener such as set screw 17. To bind the shock absorbing assembly 3 to the ankle joint housing 2, a hole 18 in the extension rod 15 can utilize a cotter pin 19. The length of the assembly is calculated such that there is a small amount of pre-load to keep the assembly taut even when no body load is applied by the user. The heel component 15 has a length such that it allows for an aesthetic foot shell cover to be attached. Additional elastomeric components may be used such as Sandwich 20, rod bumper 21, and pin bumper 22 to prevent metallic components from scraping one another to avoid noise or scratches. The ankle joint housing 2 allows for multi-axial motion with stiffness modulation in single gait cycle. During the transition from stance phase to swing phase of ambulation, and throughout the duration of the swing phase, the dorsiflexion angle is actively increased without the user's input. This actively induced dorsiflexion angle is 7 degrees (measured from neutral). Additionally, during the stance phase, plantarflexion is allowed up to 5 degrees (measured from neutral) for a total range of motion of 12 degrees. The sagittal rotation is accomplished via a pin and hole connection from a pair of connectors 23 and 24 to ankle joint housing 2. Additionally, the active dorsiflexion is enabled via an elastomeric spring 25 with notches that allow it to attach to the spring holder 26. The bottom of the elastomeric spring 25 is then trapped between spring holder 26 and the top of the connecter 23 such that it will not come loose. The spring holder 26 may be fastened to the ankle joint housing 2 via one or more fasteners such as countersunk screw 27, along with an aesthetic cover 28. Additional components and features provide boundary conditions to limit the motion and prevent noise such as plantar bumper 29, bottom bumper 30, overload protector 31, and overload bumper 32. The overload bumper 32 may be fastened to the ankle joint via a countersunk screw 27 along with an aesthetic cover 28. The prosthetic foot system 6 can be attached to the ankle joint 2 via one or more fasteners 33 and aesthetic covers 34. Additionally, elastomeric bearings 35 can be placed between the connectors 23 and 24 and the ankle joint 2 to further prevent scraping. An axial rotational sub-assembly 5 allows vertical twisting motion between the foot and pelvis during ambulation. This medial and lateral rotation is up to 15 degrees each (from neutral), for a total range of motion of 30 degrees. This sub-assembly could consist of one or more spring members 36, 37, and 38, each separated by an elastomeric piece 39, 40, and 41 to prevent scraping. A cutout in the middle of the components allows for the aforementioned extension rod 15 to pass through and connect the entire assembly within one concentric, co-located component.

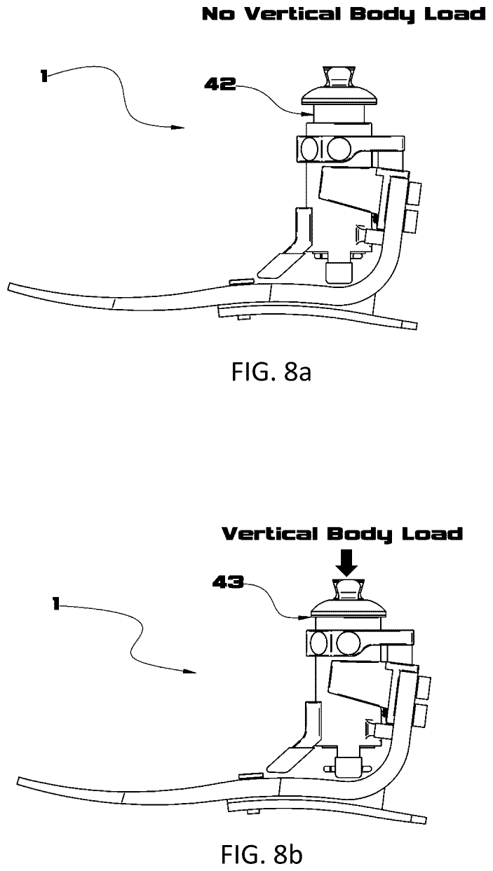

The patient's body load and initial heel strike impulse causes the shock absorber 16 to compress vertically, absorbing the shock of the initial stage of stance phase starting from initial heel-strike. The vertical loading pylon enables controlled motion by displacing up to 10 millimeters (patient load/impulse dependent) to provide shock absorbing functionality. This motion is achieved in this embodiment via a polyester and rubber blend compression spring. FIG. 8a, relating to embodiment 1, shows the device's physical state with no load applied, such as in swing phase. This leads to a fully uncompressed pylon state 42. FIG. 8b, relating to embodiment 1, shows the device's physical state with body load applied, such as in initial heel strike to stance phase. With sufficient body lead, this leads to a partially or fully compressed pylon state 43. When body load and initial heel strike impulse are applied, the shock absorber 16 compresses to allow the controlled motion. A J-shaped, energy storing keel 13 is present. The keel is able to invert/evert and plantar/dorsi-flex when the relevant body load is applied. The device's keel can be made of carbon fiber composite, a material widely used in industry for its energy storing properties. Upon load application starting from heel strike, the keel will bend like a spring, and during toe push off transitioning into swing phase, the keel will release the stored energy to return to its original shape.

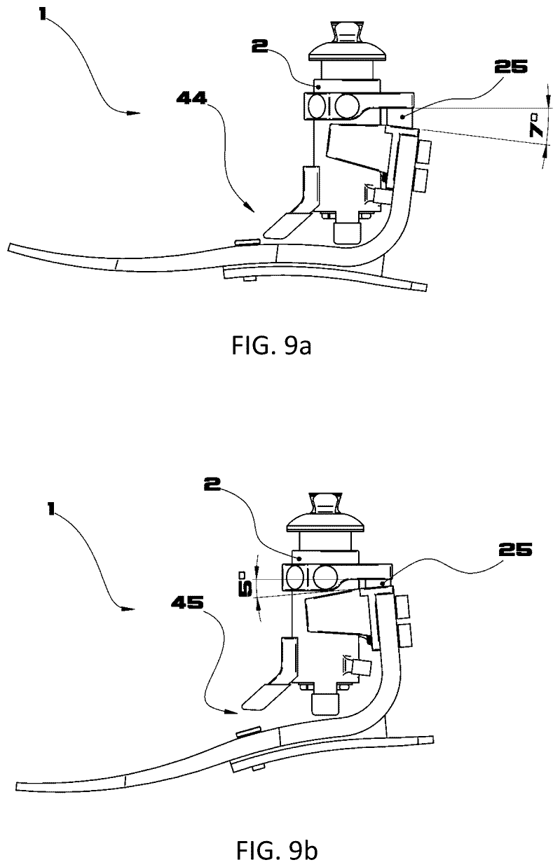

The device contains an articulating ankle joint component 2 that provides multi-axial motion with stiffness modulation in single gait cycle. This component's holes rotate around the pin joints on symmetrical lateral connectors 23 and 24. It utilizes a pair of symmetrical rotating pin and hole connectors. The device utilizes a sponge-like elastomeric structure 25 that functions as a compliant spring such that at transition of stance phase to swing phase, the product will increase the ankle's dorsiflexion angle and maintain it throughout swing phase. This component is easily overpowered by body weight and compresses during stance phase to allow plantarflexion. FIG. 9a, relating to embodiment 1, illustrates the device in a dorsiflexion state 44 of 7°, and FIG. 9b, relating to embodiment 1, illustrates the device in a plantarflexion state 45 of 5°. During swing phase, the dorsiflexion is actively enforced by the elastomeric spring 25. The TPU spring will also, due to its interaction with body load, automatically adjust the heel height/angle and range of motion to accommodate the patient's shoe selection. Further manual adjustment of the range of motion and heel height is also available via choosing an elastomeric spring that is of the desired density/size which will change the mechanical characteristics such as stiffness, allowable range of motion, and default dorsiflexion angle. The articulating component exhibits non-linear stiffness in the sagittal plane. The elastomeric spring 25 enforcing dorsiflexion is very weak and easy to overpower by body load during initial heel strike. However, towards the end of stance phase as toe-push off begins to occur and the dorsiflexion reaches its maximum and the keel 13 bends under load, the overload protector 31 engages against the keel 13 and causes the articulating ankle joint 2 to exhibit relatively high stiffness to allow for a stable and strong toe-push off. Additionally, this stiffener significantly shortens the effective cantilever length of the keel 13, which significantly increases its stiffness dynamically.

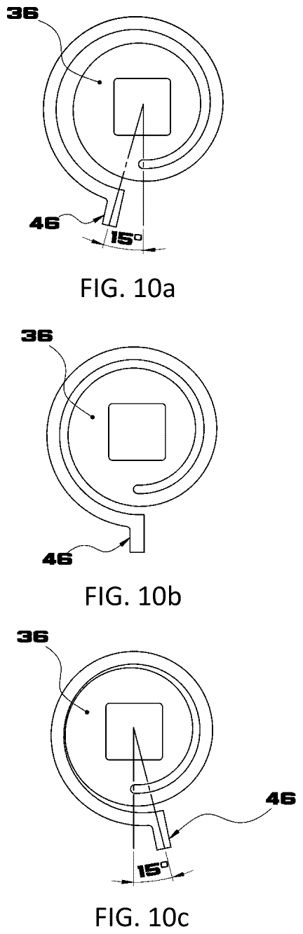

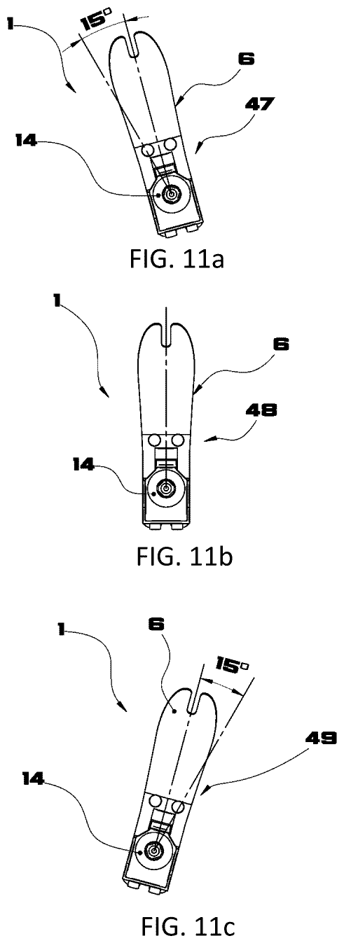

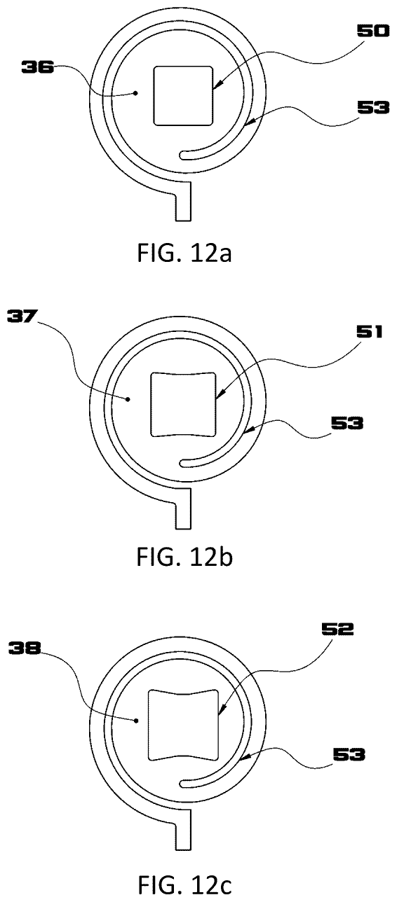

The device contains an axial rotation sub-assembly 5 that allows for vertical twisting motion between the foot and pelvis during ambulation. The axial rotation sub-assembly could consist of 3 stacked torsional springs each separated by noise cancelling elastomer to form an overall “sandwich” type component. FIG. 10, related to embodiment 1, illustrates the concept of the axial rotation via one of the torsional springs 36, with FIG. 10a representing axial deformation due to medial load, FIG. 10b representing no axial deformation due to no axial load, and FIG. 10c representing axial deformation due to lateral load. When medial or lateral force is applied, the springs enable the patient's leg to be rotated relative to the keel. Each of the torsional springs has a jutted “key” 46, which mates with a slot in the ankle joint 2 to control and restrain the motion. Because these are springs, they will return to neutral as in FIG. 10b, 0 degrees of rotation, when no load is applied, typically during swing phase. When the prosthetic foot system 6 is in stance phase, the axial rotation sub-assembly is allowed to rotate independently of the foot, allowing for vertical twisting motion between the foot system 6 and pylon 14. In FIG. 11, related to embodiment 1, FIG. 11a shows the device in a state of medial rotation 47, FIG. 11b shows the device in a state of no axial rotation 48, and FIG. 11c shows the device in a state of lateral axial rotation 49. The rotation shown is 15 degrees in the medial and lateral directions (from neutral), for a total range of motion of 30 degrees. The torsional springs exhibit differential, non-linear stiffness, that is, it will require more force to go from 14° to 15° than from 0° to 1° of rotation measured from neutral. This is due to the structural nature of the torsional springs themselves, as well as the variable engagement of the torsional springs. The first torsional spring can be made the tallest, and therefore the stiffest, while subsequent torsional springs can be made smaller. This will let additional torsional springs add stiffness while not increasing it dramatically to an uncomfortable level for the patient. The first torsional spring 36 has a tighter cutout than subsequent ones which would have more and more hourglass shaped cutouts to modulate the stiffness appropriately. In the presented embodiment, key breakpoints at 5° and 10° (in either direction) will cause a different number of torsional springs to engage, which will modulate the stiffness to higher values as the angle of rotation increases. The first torsion spring 36 has a feature such that it is always engaged in force application towards neutral. From 0° to 5° measured from neutral (in either direction), it is the only spring engaged, and the rotational stiffness is the lowest. The second torsion spring 37 has a feature such that it is engaged from 5° onwards in either direction from neutral. From 5° to 10° measured from neutral (in either direction), the first and second torsion springs 36 and 37 are engaged and the rotational stiffness is increased. The third torsion spring 38 has a feature such that it is engaged from 10° onwards in either direction from neutral. All of the torsion springs 36, 37, and 38 are engaged at 10 to 15° (in either direction), and the rotational stiffness is at its highest. FIG. 12 reveals how the concentric cutout for the extension rod 15 can be manufactured in a particular shape to allow this non-linearity. The first torsion spring 36 in FIG. 12a has a relatively square cutout 50 and will always be in contact with the extension rod 15 as it rotates. The second torsion spring 37 in FIG. 12b has a mildly hourglass shaped cutout 51 and will only be in contact with the extension rod 15 after it rotates a prescribed amount. The third torsion spring 38 in FIG. 12c, has the most hour-glass shaped cutout 52, and any subsequent springs can have more and more hourglass shaped cutouts such that they will only engage in stiffness modulation after a certain rotational angle is reached, making the stiffness dynamic. The spiral shaped cutout 53 in each of the springs 36, 37, and 38 enables the axial rotation when load is applied medially or laterally.

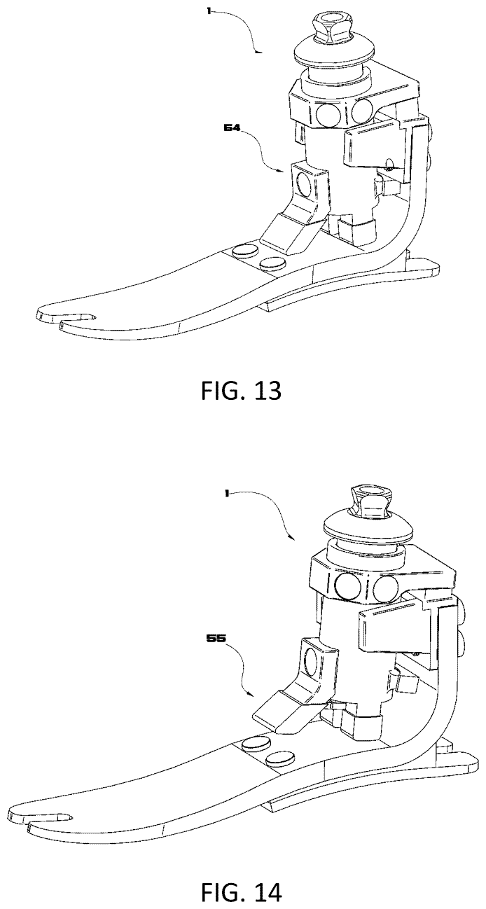

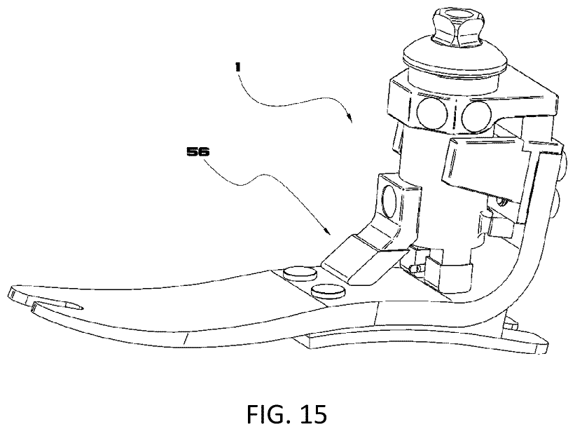

All of the aforementioned functionalities can be activated simultaneously or asynchronously depending on the state of the forces being applied on the foot. FIG. 13, relating to embodiment 1, shows the swing phase state of the foot 54, where the sagittal rotation is in dorsiflexion, the vertical loading pylon is not compressed, and the axial rotation is at neutral, or zero degrees. FIG. 14, relating to embodiment 1, shows one example of an initial heel strike scenario 55, where, due to the ground reaction force being applied primarily at the heel, the sagittal rotation is in plantarflexion, the vertical loading pylon has a small amount of initial compression, and some medial axial rotation is present. FIG. 15, relating to embodiment 1, shows an example of a scenario 56 between mid-stance and toe-off, where, due to the ground reaction force being applied primarily at the toe, the sagittal rotation is in dorsiflexion, the vertical loading pylon has a large amount of compression, and some lateral axial rotation is present. Once toe-off has occurred, and the foot is in the air without any ground reaction forces, it will return to the state 54.

As can be appreciated, embodiments can relate to a prosthetic foot system. The prosthetic foot system can include an ankle joint housing. The ankle joint housing can be configured for co-locating a rotation sub-assembly, a torsional shock absorbing sub-assembly, and/or a vertical shock absorbing sub-assembly. The prosthetic foot system can include a foot component. The foot component can be attached to the ankle joint housing. The prosthetic foot system can be configured as a co-designed architecture. For instance, the prosthetic foot system can be configured to functionally integrate at least two functions of: (i) torsional shock absorption, (ii) multi-axial motion (e.g., dorsiflexion motion, plantarflexion motion, and/or sagittal rotation) with stiffness modulation in single gait cycle, (iii) active dorsiflexion, and (iv) vertical shock absorption. This can be achieved by the interaction of the rotation sub-assembly, a torsional shock absorbing sub-assembly, and/or a vertical shock absorbing sub-assembly causing at least two of the said functions to operate in concert. The concerted action is described in detail above. An exemplary concerted action can involve the ankle joint housing providing multi-axial motion with stiffness modulation in single gait cycle in concert with the foot component providing inversion, eversion, plantar-flexing, and/or dorsi-flexing. Embodiments can provide dorsiflexion motion range between 0 degree and 7 degrees, plantarflexion motion range between 0 degrees and 5 degrees, and sagittal rotation range between 0 degrees and 16 degrees.

An exemplary embodiment of the prosthetic foot system can include a shock absorbing sub-assembly. The shock absorbing sub-assembly can include an energy storing keel and a vertical loading pylon. The prosthetic foot system can include an axial rotational sub-assembly attached to the shock absorbing sub-assembly. The prosthetic foot system can include a heel component attached to the energy storing keel. In some embodiments, the energy storing keel can be J-shaped. In some embodiments, a shock absorbing bumper can be disposed between the heel component and the energy storing keel. The shock absorbing sub-assembly and axial rotational sub-assembly can be interconnected via the extension rod to facilitate a co-locating, co-designed architecture which functionally integrates at least two functions of: (i) torsional shock absorption, (ii) multi-axial motion with stiffness modulation in single gait cycle, (iii) active dorsiflexion, and (iv) vertical shock absorption by causing the at least two functions to operate in concert.

One of the aspects that facilitates the co-designed architecture and the concerted functionality, is a torsional spring arrangement. For instance, the axial rotational sub-assembly can include plural torsional springs. The plural torsional springs can be configured to provide non-linear stiffness as rotation of the axial rotational sub-assembly occurs. In an exemplary embodiment, the plural torsional springs can include a first tortional spring, a second tortional spring, and a third tortional spring. More or less torsional springs can be used. With the exemplary embodiment, the first tortional spring can have a stiffness (S1), the second tortional spring can have a stiffness (S2), and the third tortional spring can have a stiffness (S3). It is contemplated for S1>S2>S3. This can be achieved by the plural torsional springs including plural cut-outs configured to engaged with the extension rod. (See, e.g., FIGS. 10 and 12). For instance, the first tortional spring can have a first cut-out, the second tortional spring can have a second cut-out, and the third tortional spring can have a third cut-out. The first cut-out, the second cut-out, and the third cut-out can be configured to facilitate selective engagement of the first tortional spring, the second tortional spring, and the third tortional spring, respectively, with the extension rod.

The extension rod has a cross-sectional shape. The plural cut-outs each can have a profile that corresponds to the cross-sectional shape. For instance, the first cut-out's profile can complement the cross-sectional shape to a first degree (D1). The second cut-out's profile can complement the cross-sectional shape to a second degree (D2). The third cut-out's profile can complement the cross-sectional shape to a third degree (D3). It is contemplated for D1>D2>D3. In an exemplary embodiment, the extension rod's cross-sectional shape is square, the first cut-out's profile is square, the second cut-out's profile is a concave quadrilateral with a curved side, and the third cut-out's profile is a concave quadrilateral with a curved side. A degree of concavity for the curved side of the third cut-out is greater than a degree of concavity for the curved side of the second cut-out.

The plural torsional springs can include plural cut-outs configured to engaged with the extension rod, wherein each individual cut-out can be formed in a central portion of an individual tortional spring. In addition, plural spiral shaped cut-outs can be formed and configured to facilitate axial rotation of the axial rotational sub-assembly, wherein each individual spiral shaped cut-out can be formed in a peripheral portion of an individual tortional spring.

As can be appreciated, embodiments can relate to a method for functionally integrating ambulatory functions of a prosthetic foot system. The method can involve using an embodiment of the prosthetic foot system can causing at least two functions of: (i) torsional shock absorption, (ii) multi-axial motion with stiffness modulation in single gait cycle, (iii) active dorsiflexion, and (iv) vertical shock absorption by causing the at least two functions to operate in concert.

Embodiments can also relate to providing ambulation via a prosthetic foot system. The method can involve using an embodiment of the prosthetic foot system and, during ambulation, deflecting and storing energy during an initial gait stage of a gait phase. The method can further involve releasing energy during a subsequent gait stage of the gait phase. The method can further involve providing dynamic stiffness within and/or between a swing phase, a stance phase, a toe off phase, a mid swing phase, a midstance phase, a terminal stance phase, an early flatfoot phase, a loading response phase, a pre swing phase, a swing phase, a gait phase, and a late swing phase. The method can further involve providing differential axial rotation.

It should be understood that the disclosure of a range of values is a disclosure of every numerical value within that range, including the end points. It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement.

It will be apparent to those skilled in the art that numerous modifications and variations of the described examples and embodiments are possible considering the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

It should be understood that modifications to the embodiments disclosed herein can be made to meet a particular set of design criteria. Therefore, while certain exemplary embodiments of the systems, compositions, materials, apparatuses, and methods of using and making the same disclosed herein have been discussed and illustrated, it is to be distinctly understood that the invention is not limited thereto but may be otherwise variously embodied and practiced within the scope of the following claims.

What is claimed is:

1. A prosthetic foot system, comprising:
an ankle joint housing co-locating a rotation sub-assembly, a torsional shock absorbing sub-assembly, and a vertical shock absorbing sub-assembly; and
a foot component attached to the ankle joint housing;
wherein the prosthetic foot system is configured as a co-designed architecture to functionally integrate at least two functions of: (i) torsional shock absorption, (ii) multi-axial motion with stiffness modulation in single gait cycle, (iii) active dorsiflexion, and (iv) vertical shock absorption by causing the at least two functions to operate in concert.

2. The prosthetic foot system of claim 1, wherein:
the multi-axial motion includes dorsiflexion motion, plantarflexion motion, and/or sagittal rotation.

3. The prosthetic foot system of claim 1, wherein:
the ankle joint housing provides multi-axial motion with stiffness modulation in single gait cycle in concert with the foot component providing inversion, eversion, plantar-flexing, and dorsi-flexing.

4. The prosthetic foot system of claim 3, wherein:
the dorsiflexion motion range is between 0 degree and 7 degrees;
the plantarflexion motion range is between 0 degrees and 5 degrees; and
the sagittal rotation range is between 0 degrees and 16 degrees.

5. A prosthetic foot system, comprising:
a shock absorbing sub-assembly including an energy storing keel and a vertical loading pylon;
an axial rotational sub-assembly attached to the shock absorbing sub-assembly;
a heel component attached to the energy storing keel; and
an extension rod;
wherein the shock absorbing sub-assembly and axial rotational sub-assembly are interconnected via the extension rod; and
wherein the prosthetic foot system is configured as a co-designed architecture to functionally integrate at least two functions of: (i) torsional shock absorption, (ii) multi-axial motion with stiffness modulation in single gait cycle, (iii) active dorsiflexion, and (iv) vertical shock absorption by causing the at least two functions to operate in concert.

6. The prosthetic foot system of claim 5, wherein:
the energy storing keel is J-shaped.

7. The prosthetic foot system of claim 5, further comprising:
a shock absorbing bumper is disposed between the heel component and the energy storing keel.

8. The prosthetic foot system of claim 5, wherein:
the axial rotational sub-assembly includes plural torsional springs configured to provide non-linear stiffness as rotation of the axial rotational sub-assembly occurs.

9. The prosthetic foot system of claim 8, wherein:
the plural torsional springs includes a first tortional spring, a second tortional spring, and a third tortional spring; and
the first tortional spring has a stiffness (S1), the second tortional spring has a stiffness (S2), and the third tortional spring has a stiffness (S3), wherein S1>S2>S3.

10. The prosthetic foot system of claim 9, wherein:
the plural torsional springs includes plural cut-outs configured to engaged with the extension rod.

11. The prosthetic foot system of claim 10, wherein:
the first tortional spring has a first cut-out, the second tortional spring has a second cut-out, and the third tortional spring has a third cut-out; and
the first cut-out, the second cut-out, and the third cut-out are configured to facilitate selective engagement of the first tortional spring, the second tortional spring, and the third tortional spring, respectively, with the extension rod.

12. The prosthetic foot system of claim 11, wherein:
the extension rod has a cross-sectional shape;
the plural cut-outs each have a profile that corresponds to the cross-sectional shape;
the first cut-out's profile complements the cross-sectional shape to a first degree (D1), the second cut-out's profile complements the cross-sectional shape to a second degree (D2), and the third cut-out's profile complements the cross-sectional shape to a third degree (D3); and
D1>D2>D3.

13. The prosthetic foot system of claim 12, wherein:
the extension rod's cross-sectional shape is square;
the first cut-out's profile is square;
the second cut-out's profile is a concave quadrilateral with a curved side;
the third cut-out's profile is a concave quadrilateral with a curved side; and
a degree of concavity for the curved side of the third cut-out is greater than a degree of concavity for the curved side of the second cut-out.

14. The prosthetic foot system of claim 9, wherein:
the plural torsional springs includes:
plural cut-outs configured to engaged with the extension rod, each individual cut-out being formed in a central portion of an individual tortional spring; and
plural spiral shaped cut-outs configured to facilitate axial rotation of the axial rotational sub-assembly, each individual spiral shaped cut-out being formed in a peripheral portion of an individual tortional spring.

15. A method for functionally integrating ambulatory functions of a prosthetic foot system comprising: an ankle joint housing including a rotation sub-assembly, a torsional shock absorbing sub-assembly, and a vertical shock absorbing sub-assembly; and a foot component attached to the ankle joint housing, the method comprising:
causing at least two functions of: (i) torsional shock absorption, (ii) multi-axial motion with stiffness modulation in single gait cycle, (iii) active dorsiflexion, and (iv) vertical shock absorption by causing the at least two functions to operate in concert.

16. A method for providing ambulation via a prosthetic foot system comprising: an integrated ankle joint housing including a rotation sub-assembly, a torsional shock absorbing sub-assembly, and a vertical shock absorbing sub-assembly; and a foot component; and wherein: the integrated ankle joint housing is attached to the foot component; the integrated ankle joint housing is configured to provide multi-axial motion; and the foot component is configured to invert, evert, plantar-flex, and dorsi-flex, the method comprising:
during ambulation:
deflecting and storing energy during an initial gait stage of a gait phase; and
releasing energy during a subsequent gait stage of the gait phase.

17. The method of claim 16, further comprising:
during ambulation:
providing dynamic stiffness within and/or between a swing phase, a stance phase, a toe off phase, a mid swing phase, a midstance phase, a terminal stance phase, an early flatfoot phase, a loading response phase, a pre swing phase, a swing phase, a gait phase, and a late swing phase.

18. The method of claim 16, further comprising:
during ambulation:
providing differential axial rotation.
